(19) United States
(12) Patent Application Publication
(10) Pub. No.: US 2024/0060998 A1
(43) Pub. Date: Feb. 22, 2024

(54) A METHOD TO IDENTIFY LVAD PATIENTS WITH ELEVATED LEVELS OF BLOOD ACTIVATION USING COUPON TESTS

(71) Applicant: Medtronic, Inc., Minneapolis, MN (US)

(72) Inventors: Narendra K. Simha, Falcon Heights, MN (US); Sinduja Thinamany, Blaine, MN (US); James W. Dawson, Minneapolis, MN (US)

(21) Appl. No.: 18/260,914

(22) PCT Filed: Feb. 1, 2022
(86) PCT No.: PCT/US2022/014644

§ 371 Date: Jul. 10, 2023

Related U.S. Application Data
(60) Provisional application No. 63/144,017, filed on Feb. 1, 2021.

(51) Int. Cl. G01N 33/86; G01N 1/28

(52) U.S. Cl. CPC G01N 33/86; G01N 1/28; G01N 2333/974; G01N 2333/8128; G01N 2800/226; G01N 2800/50

(57) ABSTRACT

A method for determining a patient's likelihood of experiencing a thromboembolic event when receiving an implantable blood contacting medical device. The method includes extracting a sample of blood from the patient. The sample of blood is exposed to a metal, metal alloy, or ceramic in a test tube. The sample of the blood is agitated in the test tube. A thromboembolic marker for the sample in the test tube is measured. If the thromboembolic marker for the sample in the test tube is higher than a predetermined thromboembolic marker threshold, it is determined that the patient is likely to experience the thromboembolic event when receiving the blood contacting implantable medical device.

### FIELD

The present technology is generally related to coupon assays for determining the likelihood of developing thrombus when receiving an implantable medical device.

### BACKGROUND

Implantable blood pumps, such as Ventricular Assist Devices (VADs), survivability at two years is comparable to that of transplanted hearts, but VAD patients have a considerably higher incidence of thrombo-embolic adverse events. VAD patient medications, for example, anti-coagulants, are titrated based on prothrombin time (PT), which is a test used to help detect and diagnose a bleeding disorder or excessive clotting disorder. An international normalized ratio (INR) is calculated from a PT result and is utilized to monitor the effectiveness of blood-thinning medication. However, some VAD patients experience thrombo-embolic events while others have bleeding complications, although both groups may have the same INR levels.

### SUMMARY

The techniques of this disclosure generally relate to coupon assays for determining the likelihood of developing thrombus when receiving an implantable blood pump.

In one aspect, the present disclosure provides a method for determining a patient's likelihood of experiencing a thromboembolic event when receiving an implantable blood contacting medical device. The method includes extracting a sample of blood from the patient. The sample of blood is exposed to a metal, metal alloy, or ceramic in a test tube. The sample of the blood is agitated in the test tube. A thromboembolic marker for the sample in the test tube is measured. If the thromboembolic marker for the sample in the test tube is higher than a predetermined thromboembolic marker threshold, it is determined that the patient is likely to experience the thromboembolic event when receiving the blood contacting implantable medical device.

In another aspect of this embodiment, the sample of blood is 5 ml or less.

In another aspect of this embodiment, the sample of blood is agitated on a roller for a predetermined period of time.

In another aspect of this embodiment, the predetermined period of time is 5, 30, or 60 minutes.

In another aspect of this embodiment, the sample of blood is extracted before the patient receives the implantable medical device.

In another aspect of this embodiment, the sample of blood is extracted after the patient receives the implantable medical device.

In another aspect of this embodiment, the thromboembolic marker threshold is an average thromboembolic marker level measured from a plurality of donor samples exposed to the metal, metal alloy, or ceramic and agitated for a predetermined period of time.

In another aspect of this embodiment, the metal, metal alloy, or ceramic is Titanium or Titanium Nitride.

In another aspect of this embodiment, the method further includes measuring a thromboembolic marker level of a second sample of blood from the patient in fluid communication with an implantable medical device and comparing the measured thromboembolic marker level of the second sample with the sample of blood exposed to the metal, metal alloy, or ceramic.

In another aspect of this embodiment, the method further includes determining that the measured thromboembolic marker levels in the second sample of blood is indicative or not indicative of the likelihood of experiencing the thromboembolic event when receiving the blood contacting implantable medical device based on the comparison.

In one aspect, a method for determining a patient's likelihood for experiencing a thromboembolic event when receiving an implantable blood pump includes extracting a plurality of samples of blood from the patient. The plurality of samples of blood is separately exposed to titanium (Ti) and titanium nitride (TiN) in respective test tubes. Each of the plurality of samples of the blood in the respective test tubes is agitated. Thrombin-antithrombin complex (TAT) levels are measured for each of the plurality of samples in the respective test tubes. If the TAT levels in the respective test tubes containing Ti and TiN are higher than predetermined TAT thresholds for Ti and TiN exposed blood in respective test tubes, it is determined that the patient is likely to experience the thromboembolic event when receiving the implantable blood pump.

In another aspect of this embodiment, each of the plurality of samples of blood is 5 ml or less.

In another aspect of this embodiment, each of the plurality of samples of blood is agitated on a roller for a predetermined period of time.

In another aspect of this embodiment, the predetermined period of time is one of 5 minutes, 30 minutes, or 60 minutes.

In another aspect of this embodiment, the plurality of samples of blood is extracted before the patient receives the implantable blood pump.

In another aspect of this embodiment, the plurality of samples of blood is extracted after the patient receives the implantable blood pump.

In another aspect of this embodiment, the predetermined TAT threshold for Ti and TiN exposed blood is an average TAT level measured from a plurality of donor samples exposed to Ti and TiN and agitated for a predetermined period of time.

In another aspect of this embodiment, the method further includes measuring a TAT level of a second sample of blood from the patient in fluid communication with an implantable blood pump and comparing the measured TAT level of the second sample with the plurality of samples of blood exposed to Ti and TiN.

In another aspect of this embodiment, the method further includes determining that the measured TAT levels in the second sample of blood is indicative or not indicative of a likelihood of experiencing the thromboembolic event when receiving the implantable blood pump based on the comparison.

In one aspect, a method for determining a patient's likelihood for experiencing a thromboembolic event when receiving an implantable blood pump includes extracting a plurality of samples of blood from the patient. The plurality of samples of blood is exposed separately to titanium (Ti) and titanium nitride (TiN) in respective test tubes, a volume of blood in the respective test tubes being selected to maximize a surface area of the Ti and TiN exposed to blood. Each of the plurality of samples of the blood in the respective test tubes is agitated on a plurality of rollers for 60 minutes. Thrombin-antithrombin complex (TAT) levels for each of the plurality of samples in the respective test tubes is measured. If the TAT levels in the respective test tubes containing Ti and TiN are higher than predetermined TAT thresholds for Ti and TiN exposed blood in respective test tubes, it is determined that the patient is likely to experience a thromboembolic event when receiving the implantable blood pump.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a flow chart illustrating the steps of the present method in accordance with the present application;

FIG. 2 is an image showing exemplary coupons in a test tube;

FIG. 3 is a graph showing TAT levels for various donors for exemplary test coupons; and

FIG. 4 is a chart showing whether the TAT levels shown in FIG. 3 for the various exemplary coupons are correlated to an increased TAT levels in an HVAD sample.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

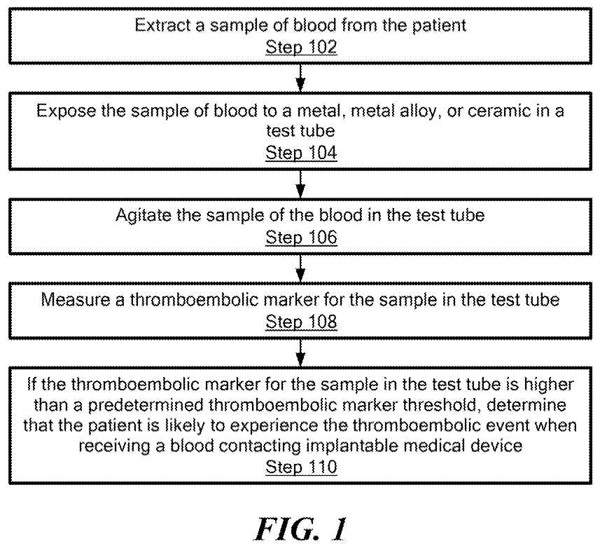

Referring now to FIG. 1 in which an exemplary method of the present application is shown. As used herein, a coupon test or coupon assay refers to samples of blood taken from one or more donors, with or without blood contacting implantable medical devices, such as implantable blood pumps, and exposed to various sample materials found in implantable medical devices for a predetermined period of time at a predetermined temperature and also exposed to an anticoagulant such as heparin at doses in the range of 0.6 unit per mL up to 2 units per mL. For example, in an exemplary configuration, the coupon test described below is used to identify the likelihood that a recipient of an implantable blood pump, which is composed of Titanium (Ti), or alloys such as Ti-6Al-4V and Ti-6Al-4V ELI, ceramics such as Titanium Nitride (TiN), or Zirconia alloys, experiences a thromboembolic event. In other configurations, the coupons may be composed other materials found in implantable medical devices, such as pacing devices, defibrillators, heart valves, frames/scaffolds for heart valves, flow diverters, coils, filters, scaffolds, stents (cardiac, thoracic, peripheral and other arterial and vein locations), dialysis and cardiopulmonary catheters and cannula. Such materials may include platinum, cobalt, chromium, nickel, or various combination of the same, magnesium, gold, silver, copper, steel, MP35N, aluminum, titanium, or various combination of the same, 316Lstainlesssteel, high nitrogen stainless steel, e.g., BIODUR108, tantalum, platinum, platinum-iridium alloy, and combinations of any of the same, Zirconia, alumina and various combinations or doped versions of the same. In addition, blood pumps may be composed of any polymer that is biocompatible and biostable. One or more of these materials may be in the form of coatings or surface modifications.

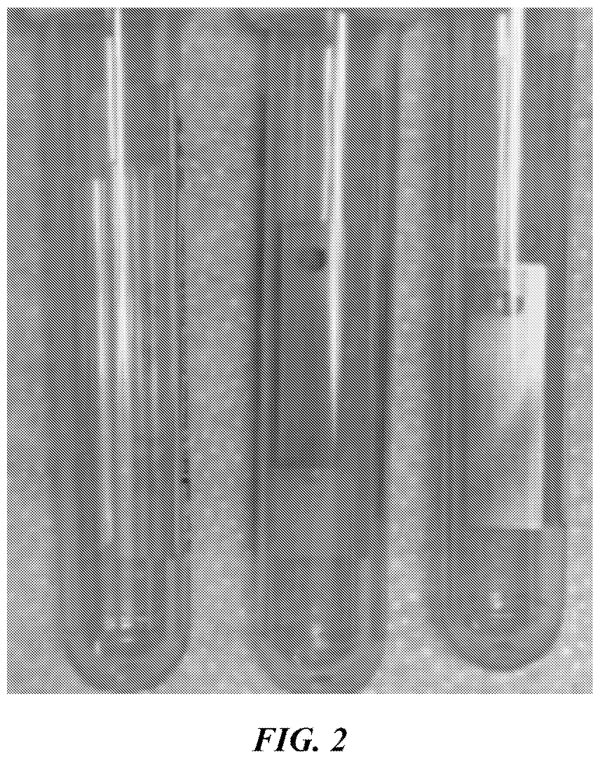

In an exemplary method, one or more samples of blood, for example, 5 ml or less per sample is extracted from a donor patient (Step 102). The donor patient may have an implanted medical device when the sample is extracted, or may receive one after the sample is extracted. Each sample of blood may be placed in a test tube for further examination. For example, as shown in FIG. 2, three samples of 3.8 ml of blood each are placed in respective test tubes containing Ti, TiN, or other metals, metal alloys, ceramics, or materials described above. The volume of blood is selected to maximize the surface area of the blood exposed to the coupon. For example, in one configuration, the surface area of the coupon to blood is approximately 1.3 cm²/mL, but may range from 1.0 cm²/mL to 2/0 cm²/mL. In particular, the blood sample is exposed to Ti or TiN separately to determine how the blood reacts to each of those materials (Step 104). For example, blood samples in the respective test tubes may be placed on rollers or otherwise rocked to agitate the blood to simulate the blood interacting with an implantable blood pump or other blood contacting implantable medical device (Step 106). The test tubes with the blood samples and coupons may be agitated for a predetermined period of time. In one embodiment, the blood samples with the respective coupons are rolled on the rollers for 5, 30, or 60 minutes at a temperature of 37 degrees Celsius. After the predetermined time period, the blood samples may be centrifuged and the plasma extracted for analysis or the entirety of the blood may be measured. A thromboembolic marker, such as thrombin-antithrombin complex (TAT) levels, are measured in each blood sample in each respective test tube (Step 108). Other thromboembolic markers may include a measurement of thrombogram features (time to peak, peak thrombin, endogenous thrombin potential), markers for Fibrin formation, for example, Fibrin peptide, or Fibrin monomer, or markers for pro-thrombin, or markers of platelet activation (such as beta-thromboglobulin, p-selectin), or platelet-platelet microaggregates, or platelet-leukocyte microaggregates.

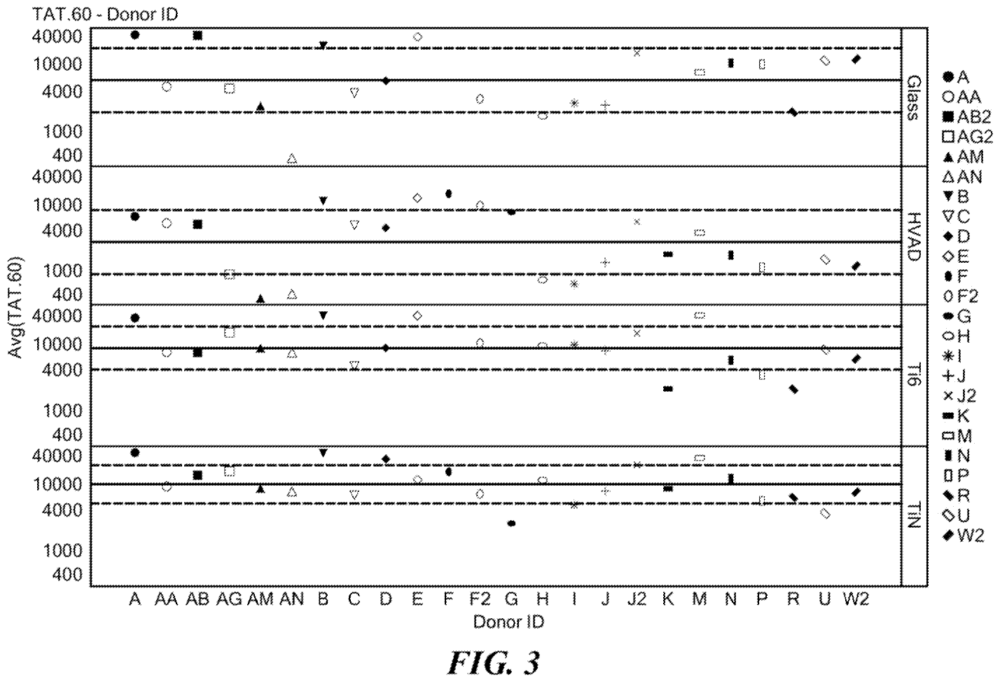

An example of the results from a plurality of sample donors is shown in FIG. 3. If the TAT levels for the sample in the test tube are higher than a predetermined TAT threshold for the metal, metal alloy, or ceramic exposed blood respectively then it is determined that the patient is likely to experience a thromboembolic event when receiving the blood contacting implantable medical device (Step 110). For example, as shown in FIG. 3, the TAT levels measured at 60 min of agitation for the plurality of donor samples are measured for several coupons, namely, Ti, TiN, and glass. Also measured are TAT levels from a blood samples from the same plurality of donors and placed in a blood loop formed with an implantable blood pump, for example, the HVAD™ pump manufactured by HeartWare, Inc. The measurements are plotted on a graph showing the average TAT levels from the plurality of donor samples exposed to coupons shown in a solid line and a standard deviation above and below the average shown in dashed lines. In general, donors with higher TAT levels for Ti and TiN also had higher TAT levels for blood in the HVAD™ loop.

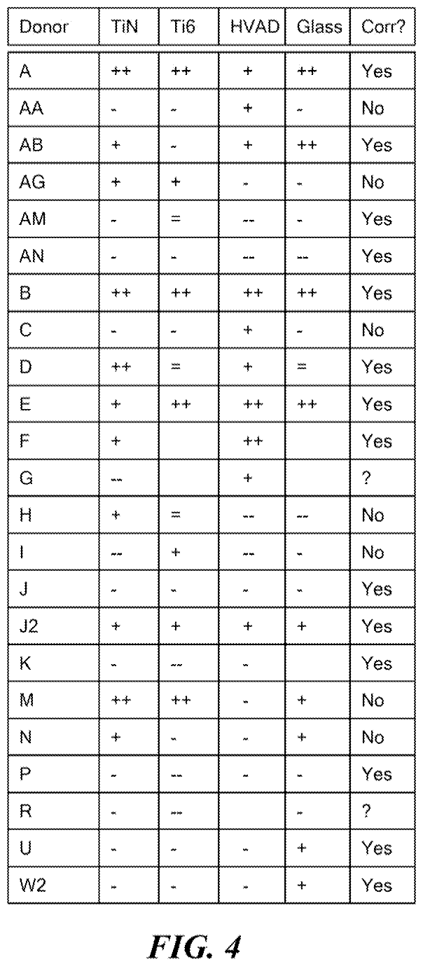

Referring now to FIG. 4, the TAT levels for the blood in the HVAD loop is compared to TAT levels in the samples exposed to metal, metal alloys, or ceramic, for example, Ti and TiN. In general, if the TAT levels in the samples of blood exposed to Ti and TiN are above average, as indicated by a “+” sign, then TAT levels in the blood samples in the HVAD loop are similarly above average, indicative of a likelihood of experiencing a thromboembolic event when receiving the blood contacting implantable medical device composed of the same materials. Moreover, if the TAT levels in the samples of blood exposed to Ti and TiN are above a standard deviation, as denoted by “++”, then similarly TAT levels in the blood samples in the HVAD loop are also a standard deviation above the average, which is indicative of a likelihood of experiencing a thromboembolic event when receiving a blood contacting implantable medical device composed of the same materials. Additionally, if the TAT levels in the samples of blood exposed to Ti and TiN are below average, as indicated by a “−” sign, then TAT levels in the blood samples in the HVAD loop are similarly below average, which is not indicative of a likelihood of experiencing a thromboembolic event when receiving the blood contacting implantable medical device composed of the same materials. Moreover, if the TAT levels in the samples of blood exposed to Ti and TiN are below a standard deviation, as denoted by “—”, then similarly TAT levels in the blood samples in the HVAD loop are also a standard deviation below the average, which is not indicative of a likelihood of experiencing a thromboembolic event when receiving a blood contacting implantable medical device composed of the same materials. Although the TAT threshold is described herein an average TAT value from a predetermined number of donor samples, it is further contemplated that the TAT threshold may be a predetermined number based on empirical data from a larger defined data set. Alternatively, the threshold may be a range of values.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

Example 1. A method for determining a patient's likelihood of experiencing a thromboembolic event when receiving an implantable blood contacting medical device, the method comprising:
extracting a sample of blood from the patient;
exposing the sample of blood to a metal, metal alloy, or ceramic in a test tube;
agitating the sample of the blood in the test tube;
measuring a thromboembolic marker for the sample in the test tube; and
if the thromboembolic marker for the sample in the test tube is higher than a predetermined thromboembolic marker threshold, determining that the patient is likely to experience the thromboembolic event when receiving the blood contacting implantable medical device.

Example 2. The method of Example 1, wherein the sample of blood is 5 ml or less.

Example 3. The method of Example 1, wherein the sample of blood is agitated on a roller for a predetermined period of time.

Example 4. The method of Example 3, wherein the predetermined period of time is 5, 30, or 60 minutes.

Example 5. The method of Example 1, wherein the sample of blood is extracted before the patient receives the implantable medical device.

Example 6. The method of Example 1, wherein the sample of blood is extracted after the patient receives the implantable medical device.

Example 7. The method of Example 1, wherein the thromboembolic marker threshold is an average thromboembolic marker level measured from a plurality of donor samples exposed to the metal, metal alloy, or ceramic and agitated for a predetermined period of time.

Example 8. The method of Example 1, wherein the metal, metal alloy, or ceramic is Titanium, Titanium Nitride or Zirconia.

Example 9. The method of Example 1, further including:
measuring a thromboembolic marker level of a second sample of blood from the patient in fluid communication with an implantable medical device; and
comparing the measured thromboembolic marker level of the second sample with the sample of blood exposed to the metal, metal alloy, or ceramic.

Example 10. The method of Example 9, further including determining that the measured thromboembolic marker levels in the second sample of blood is indicative or not indicative of the likelihood of experiencing the thromboembolic event when receiving the blood contacting implantable medical device based on the comparison.

Example 11. A method for determining a patient's likelihood for experiencing a thromboembolic event when receiving an implantable blood pump, the method comprising:
extracting a plurality of samples of blood from the patient;
exposing the plurality of samples of blood separately to titanium (Ti) and titanium nitride (TiN) in respective test tubes;
agitating each of the plurality of samples of the blood in the respective test tubes;
measuring thrombin-antithrombin complex (TAT) levels for each of the plurality of samples in the respective test tubes; and
if the TAT levels in the respective test tubes containing Ti and TiN are higher than predetermined TAT thresholds for Ti and TiN exposed blood in respective test tubes, determining that the patient is likely to experience the thromboembolic event when receiving the implantable blood pump.

Example 12. The method of Example 11, wherein each of the plurality of samples of blood is 5 ml or less.

Example 13. The method of Example 11, wherein each of the plurality of samples of blood is agitated on a roller for a predetermined period of time.

Example 14. The method of Example 13, wherein the predetermined period of time is one of 5 minutes, 30 minutes, or 60 minutes.

Example 15. The method of Example 11, wherein the predetermined TAT threshold for Ti and TiN exposed blood is an average TAT level measured from a plurality of donor samples exposed to Ti and TiN and agitated for a predetermined period of time.

Example 16. The method of Example 11, further including:
measuring a TAT level of a second sample of blood from the patient in fluid communication with an implantable blood pump; and
comparing the measured TAT level of the second sample with the plurality of samples of blood exposed to Ti and TiN.

Example 17. The method of Example 16, further including determining that the measured TAT levels in the second sample of blood is indicative or not indicative of a likelihood of experiencing the thromboembolic event when receiving the implantable blood pump based on the comparison.

Example 18. A method for determining a patient's likelihood for experiencing a thromboembolic event when receiving an implantable blood pump, the method comprising:
extracting a plurality of samples of blood from the patient;
exposing the plurality of samples of blood separately to titanium (Ti) and titanium nitride (TiN) in respective test tubes, a volume of blood in the respective test tubes being selected to maximize a surface area of the Ti and TiN exposed to blood;
agitating each of the plurality of samples of the blood in the respective test tubes on a plurality of rollers for 60 minutes;
measuring thrombin-antithrombin complex (TAT) levels for each of the plurality of samples in the respective test tubes; and
if the TAT levels in the respective test tubes containing Ti and TiN are higher than predetermined TAT thresholds for Ti and TiN exposed blood in respective test tubes, determining that the patient is likely to experience a thromboembolic event when receiving the implantable blood pump.

Example 19. A kit for determining a patient's likelihood of experiencing a thromboembolic event when receiving an implantable blood contacting medical device, the kit comprising:
a coupon test, the coupon test including a test tube containing at least one from the group consisting of a metal, metal alloy, and ceramic, the at least one from the group consisting of a metal, metal alloy, and ceramic being sized such that when blood is disposed within the test tube, the surface area of the at least one metal, metal alloy, and ceramic is greater than 1.0 cm²/mL.

Example 20. The kit of Example 19, further including an anticoagulant in the test tube, and wherein the anticoagulant has a concentration in the test tube when exposed to blood of between 0.6 units per mL to 2 units per mL of blood.

Various examples have been described. These and other examples are within the scope of the following claims.

1. A method for determining a patient's likelihood of experiencing a thromboembolic event when receiving an implantable blood contacting medical device, the method comprising:
exposing a sample of blood from the patient to a metal, metal alloy, or ceramic in a container;
agitating the sample of the blood in the container;
measuring a thromboembolic marker for the sample in the container;
determining the thromboembolic marker for the sample in the container is higher than a thromboembolic marker threshold; and
based on determining the thromboembolic marker is higher than the thromboembolic marker threshold, determining that the patient is likely to experience the thromboembolic event when receiving the implantable blood contacting medical device.

2. The method of claim 1, wherein the sample of blood is 5 ml or less.

3. The method of claim 1, wherein agitating the sample of the blood in the container comprises agitating the sample of blood on a roller for a predetermined period of time.

4. The method of claim 3, wherein the predetermined period of time is 5 minutes, 30 minutes, or 60 minutes.

5. The method of claim 1, wherein the sample of blood is extracted before the patient receives the implantable blood contacting medical device.

6. The method of claim 1, wherein the sample of blood is extracted after the patient receives the implantable blood contacting medical device.

7. The method of claim 1, wherein the thromboembolic marker threshold is an average thromboembolic marker level measured from a plurality of donor samples exposed to the metal, metal alloy, or ceramic and agitated for a predetermined period of time.

8. The method of claim 1, wherein the metal, metal alloy, or ceramic is Titanium, Titanium Nitride or Zirconia.

9. The method of claim 1, wherein the sample of blood is a first sample, the method further including:
measuring a thromboembolic marker level of a second sample of blood from the patient, the second sample of blood having been in fluid communication with the implantable blood contacting medical device; and
comparing the measured thromboembolic marker level of the second sample with the thromboembolic marker for the first sample of blood exposed to the metal, metal alloy, or ceramic.

10. The method of claim 9, further including determining, based on the comparison, that the measured thromboembolic marker level-s level in the second sample of blood is indicative or not indicative of the likelihood of experiencing the thromboembolic event when receiving the implantable blood contacting medical device.

11. A method for determining a patient's likelihood of experiencing a thromboembolic event when receiving an implantable blood pump, the method comprising:
exposing a plurality of samples of blood from the patient separately to titanium (Ti) and titanium nitride (TiN) in respective containers;
agitating each sample of the plurality of samples of the blood in the respective containers;
measuring thrombin-antithrombin complex (TAT) levels for each sample of the plurality of samples in the respective containers;
determining the TAT levels in the respective containers containing Ti and TiN are higher than respective TAT thresholds for Ti and TiN exposed blood in respective, containers; and
based on determining the TAT levels in the respective containing Ti and TiN are higher than the respective TAT thresholds, determining that the patient is likely to experience the thromboembolic event when receiving the implantable blood pump.

12. The method of claim 11, wherein each sample of the plurality of samples of blood is 5 ml or less.

13. The method of claim 11, wherein agitating each sample of the plurality of samples of blood comprises agitating each sample of the plurality of samples of blood on a roller for a predetermined period of time.

14. The method of claim 13, wherein the predetermined period of time is one of 5 minutes, 30 minutes, or 60 minutes.

15. A kit for determining a patient's likelihood of experiencing a thromboembolic event when receiving an implantable blood contacting medical device, the kit comprising:
a coupon test, the coupon test including a test tube container containing at least one from the group consisting of a metal, metal alloy, or ceramic, the at least one from the group consisting of a of the metal, metal alloy, or ceramic being sized such that when blood is disposed within the test tube container, the surface area of the at least one of the metal, the metal alloy, or the ceramic is greater than 1.0 cm²/mL.

16. The kit of claim 15, further including an anticoagulant in the container, wherein the anticoagulant has a concentration in the container when exposed to blood of between 0.6 units per mL to 2 units per mL of blood

17. The kit of claim 15, wherein the container includes a test tube.

18. The method of claim 1, wherein the container includes a test tube.

19. The method of claim 11, wherein the plurality of samples of blood is a plurality of first samples of blood, the method further including:
measuring a TAT level of a second sample of blood from the patient, the second sample having been in fluid communication with the implantable blood pump; and
comparing the measured TAT level of the second sample with the TAT levels of the plurality of samples of blood exposed to Ti and TiN.

20. The method of claim 19, further comprising, based on the comparison, determining that the measured TAT level of the second sample is indicative or not indicative of the likelihood of the patient experiencing the thromboembolic event when receiving the implantable blood pump.
